(19) United States
(12) Patent Application Publication
(10) Pub. No.: US 2004/0109905 A1
(43) Pub. Date: Jun. 10, 2004

(54) Method and composition of anthocyanin-rich berry extracts that prevents or inhibits angiogenesis and helicobacter pylori and acts as a powerful antioxidant that provides various health benefits

(76) Inventor: Debasis Bagchi, Concord, CA (US)

Correspondence Address: SHEPPARD, MULLIN, RICHTER & HAMPTON LLP, 333 SOUTH HOPE STREET, 48TH FLOOR, LOS ANGELES, CA, 90071-1448, US

(21) Appl. No.: 10/664,468

(22) Filed: Sep. 17, 2003

Related U.S. Application Data
(60) Provisional application No. 60/412,118, filed on Sep. 18, 2002.

(51) Int. Cl. A61K 35/78

(52) U.S. Cl. 424/732; 424/765

(57) ABSTRACT

A method and composition for preventing or inhibiting angiogenesis and Helicobacter pylori and providing a powerful antioxidant that provides numerous health benefits. The method involves administration of a composition incorporating specific edible berry extracts and the composition is comprised of specific edible berry extracts. The method and related composition effectively inhibit the release of growth factors that trigger angiogenesis, inhibit or prevent the growth of Helicobacter pylori and act as a powerful antioxidant with a high oxygen radical absorbance capacity (ORAC) and low cytotoxicity.

This application claims the benefit of U.S. provisional Application No. 60/412,118 filed Sep. 18, 2002.

### BACKGROUND OF THE INVENTION

The present invention relates generally to a method and related composition for providing various health benefits to humans and more particularly to a method and composition of anthocyanin-rich berry extracts that prevents or inhibits angiogenesis and Helicobacter pylori and acts as a powerful antioxidant that provides various health benefits.

Angiogenesis is the term for the formation of blood vessels in a person. Normally, angiogenesis occurs in a person as part of the process of healing from a wound, to provide blood to the area of injury. However, angiogenesis also can be prompted by tumors in a body. Angiogenesis is a key event relating to tumor growth and cancer metastases. Tumors produce large amounts of growth factors, such as vascular endothelial growth factor (VEGF), which leads to blood vessel growth in order to provide a blood supply to the tumor. VEGF plays a crucial role for the vascularization of tumors. Its release leads to tumor growth and possible flow of cancer cells into the circulatory system of the person. In particular, recent evidence suggests VEGF as the major skin angiogenic factor. Tumors produce ample amounts of VEGF, which stimulates the proliferation and migration of endothelial cells, thereby inducing tumor vascularization by a paracrine mechanism. VEGF receptors are highly expressed by the endothelial cells in tumor blood vessels. VEGF expression can be induced in various cell types by a number of stimuli, including cytokines and oxidants present at the tumor site.

As a result of the relationship between tumor formation and angiogenesis, anti-angiogenic methods to prevent and treat cancer have become an area of focus for research. These anti-angiogenic methods generally break down into two approaches. One approach focuses on specific pharmaceuticals that work to efficiently limit tumor angiogenesis. These pharmaceuticals are used to halt new blood vessel growth to cut off the blood supply to tumors. Generally, patients are provided angiogenesis inhibitors that counteract the effects of the growth factors released by the tumors. These therapies have been shown to be relatively successful in inhibiting tumor growth in patients. However, these pharmaceutical therapies are not completely successful and can cause undesirable side effects in patients. These therapies can also be harmful if they are not prescribed or administered correctly.

Other methods to limit angiogenesis focus on the use of diet-based, non-pharmaceutical, nutritive compositions. These approaches provide numerous advantages. Non-pharmaceutical methods and treatments typically have fewer side effects, can usually be taken for longer periods of time and are accepted by a larger portion of the population. While it has been shown that consumption of a plant-based diet can help prevent the development and progression of tumors associated with extensive neo-vascularization, the underlying mechanism of this method remains unclear. In any case, previous studies suggest that some cancer related events may be prevented by changes in diet.

With respect to dietary treatment approaches, the anti-angiogenic properties of edible plant products have been previously reported in a number of studies. Flavonoids, sulphated carbohydrates, or terpenoids have been suspected to be the active anti-angiogenic components of plant products. Catechins and polyphenols from plant extracts such as green tea show potent anticancer activity. Silymarin, a naturally occurring flavonoid antioxidant, exhibits anti-cancer effects against several epithelial cancers. It has been proposed that flavonoids may contribute to the preventive effect of a plant-based diet on chronic diseases, including solid tumors. Although there is a general agreement that certain plant products may posses anti-angiogenic properties, the underlying mechanisms are not well characterized. As a result, an effective non-pharmaceutical treatment for angiogenesis does not presently exist. Therefore, it should be appreciated that there exists a need for a non-pharmaceutical method or composition that prevents or inhibits angiogenesis in people.

Helicobacter pylori (H. pylori) is now recognized as an important human pathogen and carcinogen. The World Health Organization has declared H. pylori a carcinogen, predisposing infected person to gastric cancer and lymphoma. It is estimated that 50% of the world and U.S. populations are infected with H. pylori. Various gastrointestinal disorders, including chronic gastritis, gastric inflammatory diseases, peptic ulcer disease and gastric cancer have been associated with H. pylori infection.

Oxygen free radicals and oxidative stress have been implicated in several gastrointestinal diseases. They appear to be important in the development of gastrointestinal injury after intestinal ischemia and reperfusion, and after hemorrhagic shock. They have also been implicated in ischemia-reperfusion injury to the liver. In gastrointestinal inflammatory diseases such as acute pancreatitis, and inflammatory bowel diseases, oxidative stress and oxygen free radicals have been shown to play an important role. Finally, increased production of free radicals has also been demonstrated to occur during the gastrointestinal metabolism of xenobiotics, which may lead to intestinal disorders.

Elevated levels of free radicals in duodenal biopsies from patients with active duodenal ulcers have been demonstrated. Studies have shown that there is greater chemiluminescence in H. pylori positive tissue, compared to negative tissue, when samples were grouped by equivalent macroscopic or microscopic damage. In part, this difference has been accounted for by a greater neutrophil infiltration in the H. pylori positive mucosa, but when biopsy specimens with equivalent neutrophil infiltration were compared directly, H. pylori positive specimens have greater chemiluminescence than negative tissue specimens. The role of free radicals in the pathogenesis of gastric mucosal injury in cases unrelated to H. pylori infection is unclear. Therefore, the production of free radicals is associated with H. pylori positive antral infection and may be an important pathogenic mechanism. Studies have also demonstrated enhanced production of free radicals in H. pylori-induced duodenal ulceration. Interestingly, cimetidine, an H2-receptor antagonist and a widely used gastroprotective medication, is a potent hydroxyl radical scavenger, further suggesting that the production of free radicals might be an important part of the pathogenic mechanism of H. pylori.

Recent studies have demonstrated increased production of free radicals in human gastric mucosal cells following incubation with different strains of H. pylori, as evidenced by enhanced production of superoxide anion and hydroxyl radicals, and increased lipid peroxidation and DNA damage in gastric tissues. The bactericidal effects of the potent antioxidant garcinol against a pathogenic strain of H. pylori have also been shown.

A number of antimicrobial agents, including amoxicillin, tetracycline, metronidazole, clarithromycin and bismuth salts, have activity against H. pylori, but none have proven therapeutic effectiveness as single agents. In general, therapeutic regimens for H. pylori infection consist of 1-2 weeks of one or two effective antimicrobial agents plus bismuth subsalicylate or a proton pump inhibitor (lansoprazole, omeprazole, esometrazan or rabeprazole sodium). Depending on the regimen used, such therapies result in eradication rates ranging from 61% to 94% in adults. Not only are antimicrobial treatments not effective in some individuals, they are expensive and can cause adverse side effects such as diarrhea and drug allergies.

Additionally, individuals who use antimicrobial treatments typically build up a resistance to antibiotics over time. Antibiotic resistance is a major threat to treatment of many infectious diseases, including H. pylori. Recent studies have shown increasing resistance of H. pylori strains to clarithromycin, which is used to treat infections caused by this pathogen. Rates of clarithromycian resistance in H. pylori isolates from children are higher than in adults, probably due to increased exposure of children to macrolides for treatment of respiratory tract infections. Alternatives to antibiotic therapy have therefore been sought for treatment of H. pylori infections. Therefore, there exists a need for a method or composition that effectively inhibits or prevents Helicobacter pylori infection, which does not have the problems and side effects commonly associated with using antibiotic therapies.

During the past two decades, an increasing number of studies have also investigated the diverse health benefits and protective effects of anthocyanins present in various fruits and vegetables. Anthocyanins are common components of berries and their extracts, which provide pigmentation and serve as natural antioxidants. Anthocyanins are also thought to serve as anti-inflammatory and anti-mutagenic agents, and provide cardioprotection by maintaining vascular permeability. Studies have shown that supplementation with berries rich in anthocyanins are effective in reducing oxidative stress due to aging and are beneficial in reversing age-related neuronal and behavioral changes. Additionally, supplementation with anthocyanins for 6-8 months has been shown to retard age related declines in neuronal and cognitive function by improving antioxidant status. Studies suggest that the diverse health benefits that anthocyanins provide are due to their ability to provide antioxidant protection and maintain DNA integrity.

Edible berries have also been shown to posses a broad spectrum of important therapeutic and chemopreventive properties. Studies suggest that the anthocyanins found in berries reduce advancing age-induced oxidative stress and assist with neuronal and cognitive functions by promoting antioxidant status. It is also believed that anthocyanins act as anti-inflammatories and antimutagenic agents, and provide cardioprotection by maintaining vascular permeability. Different edible berries have demonstrated different specific benefits, some of which include cardiovascular, neurological, urinary tract, and ocular protection, as well as antioxidant, anti-diabetic and anti-aging properties. It is believed that the different benefits provided by the various berries are due, at least in large part, to their ability to act as powerful antioxidants. Each of the different antioxidants in edible berries are thought to provide one or more of the specific benefits described above. Because antioxidants are thought to play a large part in providing the broad spectrum of benefits associated with fruits and vegetables, providing a method or composition that provides superior antioxidant protection and minimum cytotoxicity is extremely desirable.

Despite knowledge of some of the specific benefits fruits and vegetables can provide, no satisfactory treatment or composition that prevents or inhibits angiogenesis and Helicobacter pylori and provides superior antioxidant protection, while having a low cytotoxicity, has been available. Therefore, it should be appreciated that a need exists for a method and composition that safely and effectively prevents or inhibits angiogenesis and Helicobacter pylori and acts as a powerful antioxidant that provides numerous health benefits. The present invention fulfills these needs and provides further related advantages.

### SUMMARY OF THE INVENTION

The present invention resides in a method and composition for inhibiting or preventing angiogenesis and Helicobacter pylori and acting as a powerful antioxidant that provides numerous health benefits. The method involves administration of a composition incorporating specific berry extracts that prevents or inhibits angiogenesis and Helicobacter pylori and acts as a powerful antioxidant that provides numerous health benefits. The composition is a combination of specific berry extracts that also prevents or inhibits angiogenesis and Helicobacter pylori and acts as a powerful antioxidant that provides numerous health benefits.

The method and related composition are effective at inhibiting the release of growth factors that trigger angiogenesis, inhibiting Helicobacter pylori and providing a powerful antioxidant that provides superior protection against free radicals, while having a very low cytotoxicity. In particular, specific novel combinations of berry extracts were found to prevent or inhibit angiogenesis and Helicobacter pylori and posses significantly higher oxygen radical absorbance capacity (ORAC) values than grape seed proanthocyanidin extract (GSPE) or other berry extract combinations tested. Furthermore, the cytotoxicity of the composition, as determined by the lactate dehydrogenase (LDH) leakage potential (cell viability), showed that the composition of the present invention exhibited less cytotoxicity than an equal amount of any individual berry extract tested and significantly less cytotoxicity than an equal amount of GSPE.

More particularly, in one aspect of the invention, the method of the present invention includes identifying a person suffering from, or at risk of suffering from, angiogenesis and administering an effective amount of a composition made of more than one berry extract to that person to prevent or inhibit angiogenesis in that person.

In a more detailed aspect of the method of the present invention, administering the composition of the present invention to a person reduces the amount of VEGF expressed in the person to whom the composition is administered.

In another more detailed aspect of present invention, the effective amount of the composition administered is any amount that prevents or inhibits angiogenesis or Helicobacter pylori, or any amount that provides superior antioxidant protection, which typically ranges from 18 mg/dose-270 mg/dose.

In another separate and independent aspect of the method and composition of the present invention, the cytotoxicity of the composition is lower than 0.5 LDH units/liter.

In another separate and independent aspect of the method and composition of the present invention, the berry extracts of the composition are selected from the group consisting of blueberry extract, bilberry extract, cranberry extract, elderberry extract, raspberry extract and strawberry extract.

In another separate and independent aspect of present invention, the composition is approximately 50% wild blueberry extract, approximately 35% strawberry extract, approximately 7.5% cranberry extract, approximately 2.5% raspberry seed extract, approximately 2.5% elderberry extract and approximately 2.5% wild bilberry extract by weight.

In another separate and independent aspect of the present invention, the composition is approximately 50% wild blueberry extract, approximately 25% strawberry extract, approximately 12.5% wild bilberry extract, and approximately 12.5% raspberry seed extract by weight.

In another separate and independent aspect of the invention, the method of the present invention includes administering an effective amount of composition comprised of more than one berry extract that acts as a powerful antioxidant. The composition administered has a higher oxygen radical absorbance capacity than any one berry extract used in the composition.

In another more detailed aspect of the present invention, the composition has a higher oxygen radical absorbance capacity than an equal amount of GSPE.

In another more detailed aspect of the present invention, the composition has a lower cytotoxicity than an equal amount of GSPE.

In another more detailed aspect of the invention, the composition of the present invention has an oxygen radical absorbance capacity above 40 Troxol equivalents/gm fresh weight basis.

In another separate and independent aspect of the invention, the method of the present invention includes identifying a person who would benefit from using an antioxidant with a high oxygen radical absorbance capacity.

In another separate and independent aspect of the invention, the composition of the present invention is 50% wild blueberry extract, 35% strawberry extract, 7.5% cranberry extract, 2.5% raspberry seed extract, 2.5% elderberry extract and 2.5% wild bilberry extract by weight.

In another separate and independent aspect of the invention, the composition of the present invention is 50% wild blueberry extract, 25% strawberry extract, 12.5% wild bilberry extract, and 12.5% raspberry seed extract by weight.

In another more detailed aspect of the invention, the composition of berry extracts has a higher oxygen radical absorbance capacity than both an equal amount of GSPE and an equal amount of any one berry extract.

In another separate and independent aspect of the invention, the method of the present invention includes administering an effective amount of composition comprised of more than one berry extract to prevent or inhibit; the growth of Helicobacter pylori in a person.

In a more detailed aspect of the invention, the method of the present invention improves the ability of an antibiotic to prevent or inhibit the growth of Helicobacter pylori in a person.

In a more detailed aspect of the invention, the method of the present invention improves the ability of 0.1%-5.0% concentration of clarithromycin to prevent or inhibit the growth of Helicobacter pylori in a person when compared to using the same concentration of clarithromycin alone.

In another separate and independent aspect of the invention, the composition of berry extracts comprises more than one berry extract selected from the group consisting of blueberry extract, bilberry extract, cranberry extract, elderberry extract, raspberry extract, and strawberry extract and the berry extracts are selected and proportioned relative to each other to provide a composition with a high oxygen radical absorbance capacity or a composition that effectively prevents or inhibits angiogenesis or H. pylori.

In another separate and independent aspect of the invention, the composition can further include extracts from any edible berry.

Other features and advantages of the present invention should become apparent from the following description of the preferred embodiment, taken in conjunction with the accompanying drawings, which illustrate, by way of example, the principles of the invention.

### BRIEF DESCRIPTION OF THE DRAWINGS

FIG. 1 is a graphical representation of the oxygen radical absorbance capacities (ORAC) of various compositions of berry extracts within the scope of the present invention. In FIG. 1, the compositions labeled along the X axis are comprised as follows: Mix. 1. 50% wild blueberry extract (WB), 35% strawberry extract (SB), 7.5% cranberry extract (CB), 2.5% raspberry seed extract (RS), 2.5% elderberry extract (EB), 2.5% wild bilberry extract (Bil). Mix. 2: 50% WB, 25% SB, 12.5% Bil, 12.5% RS. 3: 50% WB, 35% SB, 5% CB, 3.33% Bil, 3.33% EB, 3.33% RS. 4: 50% WB, 35% SB, 5% Bil, 3.33% EB, 3.33% CB, 3.33% RS. 5. 50% WB, 35% SB, 3.75% Bil, 3.75% EB, 3.75% CB, 3.75% RS. 6: 50% WB, 35% Bil, 3.75% EB, 3.75% SB, 3.75% CB, 3.75% RS. 7.50% WB, 25% SB, 6.25% Bil, 6.25% EB, 6.25% CB, 6.25% RS. 8: 50% WB, 25% Bil, 6.25% EB, 6.25% SB, 6.25% CB, 6.25% RS. 9: 50% WB, 25% SB, 12.5% Bil, 12.5% CB. 10: 50% WB, 35% SB, 5% EB, 3.33% Bil, 3.33% CB, 3.33% RS. 11: 50% WB, 35% SB, 5% RS, 3.33% Bil, 3.33% EB, 3.33% CB. 12: 50% WB, 10% Bil, 10% EB, 10% SB, 10% CB, 10% RS. 13: 50% WB, 25% SB, 12.5% Bil, 12.5% EB. 14: 50% WB, 35% SB, 3.75% Bil, 3.75% EB, 3.75% CB, 3.75% RS. 15: 50% Bil, 10% WB, 10% EB, 10% SB, 10% CB, 10% RS. 16: 50% WB, 25% SB, 12.5% CB, 12.5% RS. 17: 50% SB, 10% WB, 10% Bil, 10% EB, 10% CB, 10% RS. 18: 50% RS, 10% WB, 10% Bil, 10% EB, 10% SB, 10% CB. 19: 50% CB, 10% WB, 10% Bil, 10% EB, 10% SB, 10% RS. 20: 50% EB, 10% WB, 10% Bil, 10% SB, 10% CB, 10% RS. The final results (ORAC values) were calculated and expressed using Trolox equiv./gm fresh weight basis. Significantly, the single asterisk (*) above each bar of FIG. 1 denotes (p<0.05) higher when compared to other combinations and two asterisks (* *) above each bar of FIG. 1 denotes (p<0.01) different when compared to other berry extract combinations.

FIG. 2 is a graphical representation of the cytotoxicity, as measured by LDH leakage from cells to media, of various berry extracts and compositions of berry extracts within the scope of the present invention and a control and GSPE. To measure the cytotoxicity of the various berry extracts HaCaT cells were seeded at 0.15×106 cells per well/1 ml to 12-well plates. After 24 hours of growth, media was changed to serum free RPMI. Berry powder extracts (50 μg/ml) or GSPE (25 μg/ml) were added to the cells as indicated. After 24 hours media was collected for lactate dehydrogenase based in vitro toxicology assay. Significantly, the single asterisks (*) above the GSPE bar of FIG. 2 indicates that (p<0.05) higher when compared to corresponding control. Also, this experiment indicated that starting at 25 μg/ml GSPE was cytotoxic.

FIG. 3 is graphical representation showing the percentage of H. pylori inhibited by clarithromyicin after a 0.25% concentration of selected berry extracts and a 0.25% concentration of the composition of berry extracts known as Mixture 1 were exposed to the effected cells.

FIG. 4 is graphical representation showing the percentage of H. pylori inhibited by 0.25% concentration of selected berry extracts and a 0.25% concentration of the composition of berry extracts known as Mixture 1.

FIG. 5 is graphical representation showing the percentage of H. pylori inhibited by clarithromyicin after a 0.50% concentration of selected berry extracts and a 0.50% concentration of the composition of berry extracts known as Mixture 1 were exposed to the effected cells.

FIG. 6 is graphical representation showing the percentage of H. pylori inhibited by 0.50% concentration of selected berry extracts and a 0.50% concentration of a composition of berry extracts known as Mixture 1.

FIG. 7 is graphical representation showing the percentage of H. pylori inhibited by 1.00% concentration of selected berry extracts and a 1.00% concentration of a composition of berry extracts known as Mixture 1.

FIG. 8 is graphical representation showing the percentage of H. pylori inhibited by clarithromyicin after a 1.00% concentration of selected berry extracts and a 1.00% concentration of the composition of berry extracts known as Mixture 1 were exposed to the effected cells.

### DETAILED DESCRIPTION OF THE PREFERRED EMBODIMENT

The present invention resides in a method and composition for preventing or inhibiting angiogenesis and Helicobacter pylori and providing superior antioxidant protection with low cytotoxicity. The method includes administering an amount of a specified composition incorporating berry extracts to a person sufficient to prevent or inhibit angiogenesis or H. pylori or provide superior antioxidant protection in that person. The present invention is also embodied in a specified composition that similarly prevents or inhibits angiogenesis or H. pylori or provides superior antioxidant protection in a person.

The specific composition discussed above incorporates more than one berry extract that includes particular antioxidants that provide anti-angiogenic effects. It has been found that extracts from a number of berries demonstrate anti-angiogenic effects. One aspect of the present invention includes a method where a person is administered an effective amount of a composition that incorporates more than one berry extract to prevent or inhibit angiogenesis or H. pylori or provide superior antioxidant protection. In another aspect of the present invention, the method for preventing or inhibiting angiogenesis or H. pylori or providing superior antioxidant protection further includes identifying a person who has, or is at risk of having, angiogenesis or H. pylori, or a person who would benefit from superior antioxidant protection. The person identified is then administered an amount of the composition of berry extracts discussed above sufficient to prevent or inhibit the unwanted angiogenesis or H. pylori or to provide superior antioxidant protection.

Preferred known compositions for use in the methods of the present invention are available from InterHealth Nutraceuticals of Benicia, Calif. Clarithromycin (Biaxin) was obtained from Abbott laboratories, North Chicago, Ill. 60064. Unless otherwise stated, all other chemicals and reagents were obtained from Sigma Chemical Co. (St. Louis, Mo.) and were of analytical grade or the highest grade available.

It should be appreciated that the scope of the present invention includes using any type of berry that is edible by human beings and that the berries themselves, or any part of them, can be used with or instead of the berry extracts discussed herein to prevent or inhibit angiogenesis or H. pylori or to provide superior antioxidant protection or other related advantages. Additionally, the scope of the present invention includes using berries that have been grown by any means, including, but not limited to, conventionally, organically, or in the wild.

In another aspect of the present invention, the method of the present invention includes administering to a person a composition incorporating wild blueberry, strawberry, cranberry, raspberry seed, elderberry, or wild bilberry extracts or any combination thereof.

In another embodiment of the present invention, the composition of the present invention includes a composition that includes two or more berry extracts from blueberries, strawberries, cranberries, raspberries, elderberries, bilberries or any combination thereof.

In yet another aspect of the present invention, the method of the present invention includes administering to a person a composition having the following composition by weight: 50% blueberry extract, 35% strawberry extract, 7.5% cranberry extract, 2.5% raspberry extract, 2.5% elderberry extract and 2.5% bilberry extract.

In another embodiment of the present invention, the present invention includes a composition with the following composition by weight: 50% wild blueberry extract, 35% strawberry extract, 7.5% cranberry extract, 2.5% raspberry seed extracts, 2.5% elderberry extract and 2.5% wild bilberry extract. A composition with this formulation is called Mixture 1.

In yet another aspect of the present invention, the method of the present invention includes administering to a person a composition having the following composition by weight: 50% blueberry extract, 25% strawberry extract, 12.5% bilberry extract and 12.5% raspberry extract.

In another embodiment of the present invention, the present invention includes a composition with the following. composition by weight: 50% wild blueberry extract, 25% strawberry extract, 12.5% wild bilberry extract and 12.5% raspberry seed extract. A composition with this formulation is called Mixture 2.

The actual amount of the composition administered to a person as part of the method and composition of the present invention varies depending upon various factors, including, but not limited to, the person's age, physical condition and body mass. In light of these and other factors that could effect the efficacy of the composition and method, the present invention includes administration of an amount of specific composition that prevents or inhibits angiogenesis or H. pylori or provides superior antioxidant protection. As indicated above, it is believed that the effective amount of the composition is typically from 18 mg/dose-270 mg/dose.

Additionally, in accordance with the present invention, the method and composition of the present invention can further include any inert ingredients or diluents, such as sugars or fillers, commonly used in food and drug related products or presently known in the art. In accordance with the present invention, the composition of the present invention can be provided in any form presently known to those skilled in the art, including, but not limited to, dietary supplements pill, tablet, capsule, powder, lozenge, gum, or liquid. The step of administering the composition includes administering the composition as part of foods or beverages, including, but not limited to, bars, shakes, drinks, and other processed or prepared foods or beverages.

Compositions suitable for use in the method and composition of the present invention were subjected to in vivo testing for efficacy. The testing procedures and results of the testing are provided below. The tests focused on analysis of the effect of the compositions of the present invention on skin angiogenesis, H. pylori and on the oxygen radical absorbance capacity (ORAC) of the compositions.

Determining the Compositions Ability to Inhibitior Prevent, Skin Angiogenesis

The vasculature in adult skin remains normally quiescent, due to the dominant influence of endogenous angiogenesis inhibitors over angiogenic stimuli. However, skin retains the capacity for brisk initiation of angiogenesis during inflammatory skin diseases, such as psoriasis and skin cancers, such as cutaneous squamous cell carcinomas. Moreover, cyclic vascular expansion occurs during the growth phase of hair follicles. Recent evidence suggests VEGF as the major skin angiogenic factor. During skin angiogenesis, expression of VEGF is induced in epidermal keratinocytes. VEGF is a marker of tumor invasion and metastasis in squamous cell carcinomas. Therefore, testing for VEGF expression and the effects of the compositions of the present invention on the expression of VEGF relates to efficacy in preventing angiogenesis and related tumor growth.

The compositions used for testing incorporated powders of berry extracts as described above as Mixtures 1 and 2. Also, for comparison testing, a grape seed proanthocyanidin extract (GSPE) was obtained from the InterHealth Nutraceuticals. The GSPE is a natural extract containing approximately 54% dimeric, 13% trimeric and 7% tetrameric proanthocyanidins, and a small amount of monomeric bioflavonoids.

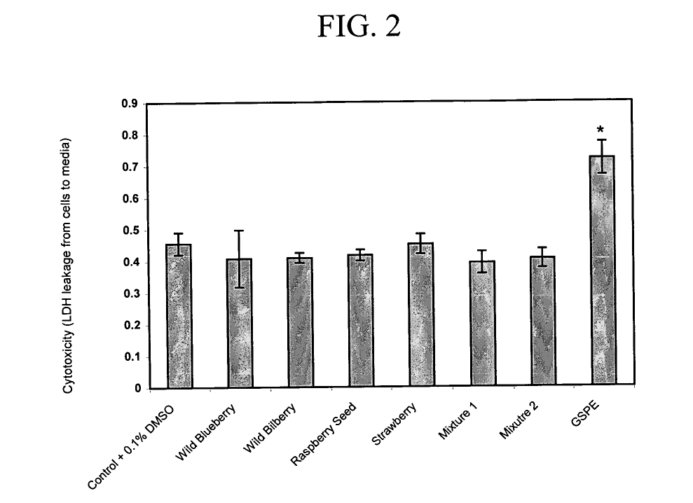

Additionally, the berry extracts were combined based on the ORAC values of the individual berry extracts. Wild blueberry and bilberry extracts demonstrated the highest ORAC value, and these were significantly higher as compared to the other berry extracts. Strawberry extract exhibited significantly higher ORAC values as compared to elderberry cranberry, and raspberry seeds. Cranberry exhibited marginally higher ORAC value compared to elderberry, while elderberry showed marginally higher ORAC value as compared to raspberry seeds. Additionally, the berry extract compositions of the present invention have a lower cytotoxcity that an equivalent amount of GSPE or an equivalent amount of any one berry extract, as shown by FIG. 2.

We initially made six combinations, which contained 50% of one berry extract and a 10% blend of the remaining extracts. Since bilberry and strawberry demonstrated higher ORAC values as compared to cranberry, elderberry and raspberry seeds, we made four combinations. The first two combinations were made using 50% wild blueberry, 25% of either wild bilberry or strawberry and 6.25% of the remaining berry extracts. The last two combinations were made using 50% wild blueberry, 35% of either bilberry or strawberry and 3.75% of the remaining berry extracts.

In another set, we made four combinations of berry extracts. The first three combinations contained 50% wild blueberry, 25% strawberry, 12.5% wild bilberry and 12.5% of either cranberry, raspberry seed or elderberry extract. The fourth combination contained 50% wild blueberry, 25% strawberry, 12.5% each of cranberry and raspberry seed extract. In the last set, we made six combinations with each containing 50% wild blueberry and 35% strawberry. The first combination also contained 3.75% each of wild bilberry, elderberry, cranberry and raspberry seed extract, while three of the combinations contained 5% of either cranberry, elderberry or raspberry seed extract, 3.33% wild bilberry extract and 3.33% each of elderberry and raspberry seed extract, or 3.33% each of cranberry and raspberry seed extract, or 3.33% each of elderberry and cranberry extract. The fifth combination also contained 5% wild bilberry and 3.33% each of elderberry, cranberry and raspberry seed extract. The last combination also contained 7.5% cranberry and 2.5% each of wild bilberry, elderberry and raspberry seed extract.

Thus, we prepared a total of twenty different combinations of berry extracts for evaluation. Table I below demonstrates the twenty different berry combinations that are within the scope of the present invention.

TABLE I
Compositions of twenty edible berry extract combinations

| | Wild | | | | | Raspberry |
|---|---|---|---|---|---|---|
| Samples | Blueberry | Bilberry | Elderberry | Strawberry | Cranberry | Seed |
| Berry Extract Composition 1 | 50% | 2.50% | 2.50% | 35.00% | 7.50% | 2.50% |
| (Mixture 1) | | | | | | |
| Berry Extract Composition 2 | 50% | 12.50% | 0.00% | 12.50% | 0.00% | 12.50% |
| (Mixture 2) | | | | | | |
| Berry Extract Composition 3 | 50% | 3.33% | 3.33% | 35% | 5% | 3.33% |
| Berry Extract Composition 4 | 50% | 5% | 3.33% | 35% | 3.33% | 3.33% |
| Berry Extract Composition 5 | 50% | 3.75% | 3.75% | 35% | 3.75% | 3.75% |
| Berry Extract Composition 6 | 50% | 35% | 3.75% | 3.75% | 3.75% | 3.75% |
| Berry Extract Composition 7 | 50% | 6.25% | 6.25% | 25% | 6.25% | 6.25% |
| Berry Extract Composition 8 | 50% | 25% | 6.25% | 6.25% | 6.25% | 6.25% |
| Berry Extract Composition 9 | 50% | 12.5% | 0% | 25% | 12.5% | 0% |
| Berry Extract Composition 10 | 50% | 3.33% | 5% | 35% | 3.33% | 3.33% |
| Berry Extract Composition 11 | 50% | 3.33% | 3.33% | 35% | 3.33% | 5% |
| Berry Extract Composition 12 | 50% | 10% | 10% | 10% | 10% | 10% |
| Berry Extract Composition 13 | 50% | 12.5% | 12.5% | 25% | 0% | 0% |
| Berry Extract Composition 14 | 50% | 3.75% | 3.75% | 35% | 3.75% | 3.75% |
| Berry Extract Composition 15 | 10% | 50% | 10% | 10% | 10% | 10% |
| Berry Extract Composition 16 | 50% | 0% | 0% | 25% | 12.5% | 12.5% |
| Berry Extract Composition 17 | 10% | 10% | 10% | 50% | 10% | 10% |
| Berry Extract Composition 18 | 10% | 10% | 10% | 10% | 10% | 50% |
| Berry Extract Composition 19 | 10% | 10% | 10% | 10% | 50% | 10% |
| Berry Extract Composition 20 | 10% | 10% | 50% | 10% | 10% | 10% |

The cells used for the test were immortalized HaCaT human keratinocytes grown in Dulbecco's modified Eagle's medium (provided by Life Technologies of Gaithersburg, Md.) and supplemented with 10% fetal bovine serum, 100 U/mL penicillin and 100 μg/mL streptomycin.

The berry extract compositions were prepared for high performance liquid chromatography (HPLC) multichannel electrochemical analysis. The test compositions were weighed out to 10 mg samples and each dissolved in 400 pA of aqueous methanol (62.5+0.29% BHA). The samples then. were ultrasonicated on ice for 2 minutes (30 s×4 pulse), and 100 JLI of 6N HCl was added to each of the samples. The samples then were bubbled with nitrogen for 30 seconds and incubated at 90° C. for 2 hours. The samples then were cooled, and 500 pA of 100% methanol was added. The samples were centrifuged at 13,000 rpm for 5 minutes at 4° C., and then were filtered using a 0.45 micron filter.

Next, the cells discussed above were tested to determine the amount of uptake of the particular constituents of the berry extracts. The cells were cultured in 150 mm×20 mm plates using conventional methods. After 24 hours of seeding, the growth media was changed to serum-free RPMI. Then, berry extracts were added in excess quantity (250 μg/mL) to allow for detection of constituents taken up by cells only in trace amounts. That is, previous experiments testing the effects of berry extracts on inducible VEGF expression have used a maximum of 50 μg/mL of berry extracts. In this case, however, a fivefold amount of the extracts from that previously used was provided to ensure that analytical limitations did not prevent detection of the presence of certain berry constituents that were taken up in low amounts. After 24 hours of this treatment, the cells were washed with PBS, scrapped and collected phosphate buffer was added to the cell pellets, and the pellets were homogenized on wet-ice and then ultrasonicated. Then, HCl (3M) was added to the samples and the resulting products were incubated for 30 min at room temperature in the dark. After this incubation, polyphenols were extracted from each sample using 2 ml of ethyl acetate and analyzed by coulometric electrochemical array detection with HPLC as indicated below.

After the preparation described above, the berry extracts, along with the GSPE extract, were tested using traditional HPLC methods. The gradient analytical system consisted of an autosampler, a thermostatic chamber and a 12-channel CoulArray detector.

The chromatography conditions are listed below:

| | |
|---|---|
| Column: | Symmetry C18 5 μm (4.6 × 250 mm) |
| Mobile phase A: | 50 mM Sodium phosphate buffer; pH 3.0; |
| | methanol (99:1 v/v) |
| Mobile phase B: | 100 mM Sodium phosphate buffer pH 3.45; |
| | acetonitrile; methanol (30:60:10 v/v/v) |
| Gradient: Conditions: | 0% B for 5 min. to 80% B by 40 min., |
| | hold at 80% B until 45 min then back |
| | to 0% B by 55 min. |
| Flow Rate: | 0.8 ml/min. |

The detector conditions were as follows:

| | |
|---|---|
| Detector: | Model 5600A, CoulArray. |
| Applied Potentials: | i) −20 to +100 mV in +80 mV increments, |
| | ii) +160 to +400 mV in 60 mV increments, |
| | iii) +500 to +700 mV in 100 mV increments |

Solutions incorporating berry extracts or GSPE were prepared fresh in dimethyl sulfoxide at concentrations such that the final concentration of the solvent in cell suspension never exceeded 0.1% prior to cell treatment, the DMSO solutions were passed through a 0.22 μM filter for sterilization. Respective controls were treated with equal volumes of dimethyl sulfoxide. The test cells were pretreated with the berry solutions.

Treatment of cells with berry compositions having concentrations of up to 50 μg/mL did not influence cell viability, as detected by a standard lactate dehydrogenase dependent viability assay. However, at a concentration of 25 μg/mL, the GSPE was toxic to the treated cells. Following incubation with the respective berry compositions, the cells were washed using a serum-free medium and then were treated with TNFα (25 ng/ml) or H₂O₂ (250 μm) in a serum-free medium.

To determine cell viability, the test cells were seeded at a density of 0.15×10⁶ cells/well/ml in 12-well plates. After 24 hours of seeding, the growth media was changed to serum-free RPMI, and berry compositions were added at a high dose of 50 μg/mL. After 24 hours, the media were collected and centrifuged at 3500 rpm for 5 minutes at 4° C. The resulting aliquots were transferred to a flat bottom plate and lactate dehydrogenase (LDH) assays were performed on them. LDH release to the media was measured using a lactate dehydrogenase-based in vitro toxicology assay kit obtained from Sigma Chemical Co. of St. Louis, Mo.

Measuring VEGF Protein

Test cells were seeded onto multiple well culture-plates. After 24 hours of growth at 80% confluency, the cells were synchronized by culturing them in a serum-deprived medium for 12 hours. Following the synchronization, the cells were treated with H₂O₂ or TNFα. The berry treatment protocols are described in the legends for Tables 2 through 4 below. A serum-free medium was selected to avoid any possible interaction between the serum components and H₂O₂. The VEGF level in the medium was determined using a commercially available ELISA kit, marketed by R and D systems of Minneapolis, Minn.

More specifically, Table 2 is a chart showing how various berry extracts and berry extract compositions, including a composition known as Mixture 1, of the present invention inhibited H₂O₂ induced expression of VEGF when compared to a control, H₂O₂ alone and GSPE and H₂O₂. Table 3 is a chart showing how various berry extracts and the berry extract compositions, including a composition known as Mixture 1, inhibited TNFα-induced expression of VEGF when compared to a control, TNFα and GSPE and TNFα. Table 4 is a chart showing how selected flavonoids and tocopherol inhibited H₂O₂-induced expression of VEGF when compared to a control and H₂O₂ alone.

TABLE II
Effect of Mixture 1 and Other Berry Extracts
on H₂O₂Induced Expression of VEGF

| Sample | VEGF (pg/ml) |
|---|---|
| Control | 76.03 ± 9.87 |
| H₂O₂ | 237.77 ± 16.60 |
| Wild Blueberry + H₂O₂ | 59.89 ± 5.56 |
| Wild Bilberry + H₂O₂ | 96.26 ± 6.57 |
| Raspberry Seed + H₂O₂ | 69.70 ± 18.24 |
| Strawberry + H₂O₂ | 88.38 ± 10.24 |
| Mixture 1 + H₂O₂ | 55.96 ± 5.66 |
| GSPE + H₂O₂ | 313.97 ± 11.57 |

HaCaT cells were seeded at density 0.45×10⁶/well/3 ml. After 24 hr, growth media was chanced to serum free RPMI and berry samples (50 μg/ml) or GSPE (25 μg/ml) were added. After 12 hours, cells were challenged with H₂O₂ (150 μM). After 12 hours of activation with H₂O₂, media was collected for ELISA. *p<0.05, higher in response to H₂O₂ treatment; *lower compared to H₂O₂ treated cells. Mean±SD of three experiments.

TABLE III
Effect of Mixture 1 and Other Berry Extracts
on TNFα Induced Expression of VEGF

| Sample | VEGF (pg/ml) |
|---|---|
| Control | 42.72 ± 0.57 |
| TNFα | 177.10 ± 21.31 |
| Wild Blueberry + TNFα | 52.89 ± 4.02 |
| Bilberry + TNFα | 96.86 ± 8.42 |
| Rasberry Seed + TNFα | 77.13 ± 15.07 |
| Strawberry + TNFα | 88.29 ± 8.07 |
| Mixture 1 + TNFα | 93.57 ± 3.48 |
| GSPE + TNFα | 236.63 ± 6.82 |

HaCaT cells were seeded at density 0.45×10⁶/well/3 ml. After 24 hr, growth media was chanced to serum free RPMI and berry samples (50 pg/ml) or GSPE (25 μg/ml) were added. After 12 hours, cells were challenged with TNFα (25 μg/ml). After 12 hours of activation with TNFα, media was collected for ELISA. *p<0.05, higher in response to TNFα treatment; *lower compared to TNFα treated cells. Mean±SD of three experiments.

TABLE IV
Effects of Selected Flavonoids and Tocopherol
on H₂O₂-Induced Expression of VEGF

| Sample | VEGF (pg/ml) |
|---|---|
| Control | 144.16 ± 26.62 |
| H₂O₂ | 380.84.77 ± 50.17 |
| α-tocopherol + H₂O₂ | 397.10 ± 64.91 |
| Ferrulic Acid + H₂O₂ | 319.19 ± 21.05 |
| Catechin + H₂O₂ | 285.89 ± 51.13 |
| Rutin + H₂O₂ | 209.06 ± 59.28 |

HaCaT cells were seeded at density 0.45×10⁶/well/3 ml. After 24 hr, growth media was chanced to serum free RPMI and either pure flavonoids (ferric acid, FA 200 nM; catechin, Cat 100 nM, ruti, rut 1 μM) at concentrations observed in berry samples or a α-tocopherol (10 μM as reference antioxidant) were added. After 12 hours, cells were challenged with H₂O₂ (150 μM). After 12 hours of activation with H₂O₂, media was collected for ELISA. *p<0.05, higher in response to H₂O₂ treatment; *lower compared to H₂O₂ treated cells. Mean±SD of three experiments.

In Vitro Angiogenesis Assay

The berry compositions were also tested to determine whether they influenced the process of angiogenesis per se. Among the various in vivo and in vitro methods for the study of angiogenesis, the in vitro Matrigel assay represents a highly reliable approach to test angiogenic or antiangiogenic properties of test species. The method is based on the differentiation of endothelial cells to form capillary like structures on a basement membrane matrix, Matrigel, derived from EHS tumor. Matrigel induces endothelial cells to differentiate as evidenced by both the morphologic changes and by the reduction in proliferation and, therefore, offers a convenient and reliable model to study biochemical and molecular events associated with angiogenesis. We used human dermal microvascular endothelial cells for this assay that was performed using a kit where the conditions are optimized for maximal capillary-like structure formation.

An in vitro angiogenesis kit marketed by CHEMICON International, Inc. of Temecula, Calif., was used for the assay. ECMatrix™ is a solid gel of basement proteins prepared from the EngelbrethHolm-Swarm (EHS) mouse tumor. The ECMatri™ (10×) solution was thawed on ice and diluted with a diluent provided with the kit. 100 μl of the diluted ECMatri™ (10×) solution was transferred to each well of a 96-well tissue culture plate and was incubated at a temperature of 37° C. for at least 1 hour to allow the matrix solution to solidify. HMVE cells then were harvested and resuspended in a media, either in the presence or absence of berry extracts. The cells, in an amount of approximately 5000 cells/well, were added on top of the solidified matrix solution and maintained in a cell culture incubator at a temperature of 37° C. overnight. Following this, Endothelial tube formation was observed and digitally photographed under an inverted light microscope at a magnification factor of 20.

Determining the Compositions Oxygen Radical Absorbance Capacity.

To determine the oxygen radical absorbance capacity (ORAC) values for the separate berry extracts and the compositions of berry extracts, samples of 25 mg of individual berry extracts each were dissolved in 1 ml of methanol. Then, 0.09 ml phosphate buffer was added to 0.01 ml of the berry/methanol solution obtained as described above. A total of 0.01 ml of each berry composition was used for this analysis.

The procedure for performing ORAC assays was performed as described in Cao, G., Alessio, H. M. and Cutler, R. G. (1993) “Oxygen-radical absorbance capacity assay for antioxidants,” Free Radic. Biol. Med. 14, 303-11, which is incorporated herein by reference. This assay measures the ability of antioxidant compounds in test materials to inhibit the decline of B-phycoerythrm (B-PE) fluorescence that is induced by a peroxyl radical generator, AAPH. The reaction mixture contained 1.6 ml of 75 mM phosphate buffer (pH of 7.0), 200 μl of 320 nM AAPH, and 100 μl of sample. Trolox, a water-soluble analog of vitamin E, was used as a control antioxidant standard. The fluorescence of B-PE was determined and recorded every 5 minutes at the excitation wavelength of 540 nm and emission wavelength of 570 nm using a Turner fluorometer until the fluorescence of the last reading declined to <5% of the first reading. The final results (ie., the ORAC values) were calculated using the differences of the areas under the quenching curves of B-PE between a blank and a sample, and they were expressed as micromoles of Trolox equivalents (TE) per gram of fresh weight.

Results

Significance between pairs of mean values was determined by Student's t test. A p<0.05 was considered significant for all analyses. Each value is the mean±the standard deviation of four replicates.

In the second set of combinations, we analyzed six combinations, which contained 50% of one berry extract and a 10% blend of the remaining five berry extracts. Results demonstrate that a combination of 50% wild blueberry with a 10% blend of the remaining five berry extracts exhibited the highest ORAC value in this set, which was marginally higher than a combination of 50% bilberry with a 10% blend of the remaining five berry extracts.

In the third set, we made four combinations. The first two combinations were made using 50% wild blueberry, 25% of either wild bilberry or strawberry and 6.25% of the remaining berry extracts. The last two combinations were made using 50% wild blueberry, 35% of either wild bilberry or strawberry and 3.75% of the remaining berry extracts. No significant difference was observed between these four combinations. Although, the fourth combination (50% wild blueberry, 35% strawberry, 3.75% each of wild bilberry, elderberry, cranberry and raspberry seed) demonstrated a slightly higher value as compared to the other three combinations.

In the fourth set, we tested four combinations. The first three combinations contained 50% wild blueberry, 25% strawberry, 12.5% wild bilberry and 12.5% of either cranberry, raspberry seed or elderberry extract. A fourth combination (designated herein as Mixture 2) contained of 25% strawberry extract, 12.5% wild bilberry extract and 12.5% raspberry seed extract, which demonstrated a marginally higher ORAC value than the combination containing 50% wild blueberry, 25% strawberry, 12.5% wild bilberry and 12.5% cranberry extracts and a significantly higher ORAC value than the remaining two combinations.

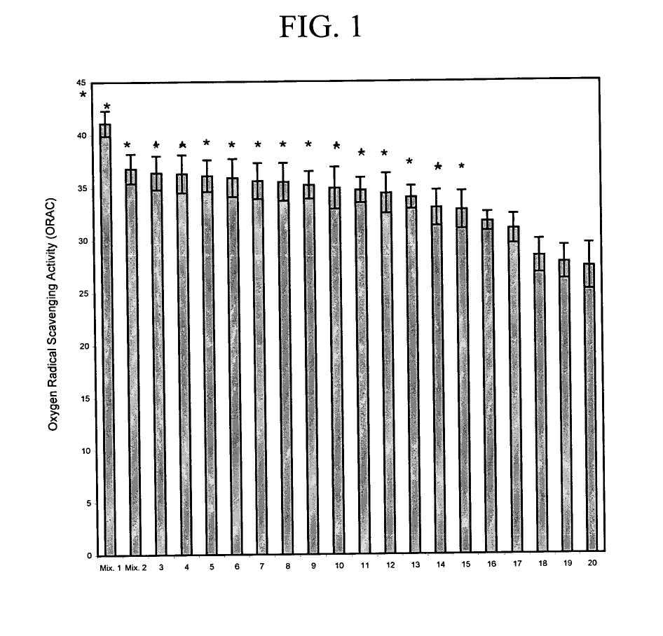

In the fifth set, we made five combinations, each containing 50% wild blueberry and 35% strawberry. The first combination also contained 3.75% each of wild bilberry, elderberry, cranberry and raspberry seed extract, while three of the combinations contained 5% of either cranberry, elderberry or raspberry seed extract, 3.33% wild bilberry extract and 3.33% each of elderberry and raspberry seed extract, or 3.33% each of cranberry and raspberry seed extract, or 3.33% each of elderberry and cranberry extract. The fifth combination also contained 5% wild bilberry and 3.33% each of elderberry, cranberry and raspberry seed extract. A sixth combination (designated herein as Mixture 1) contained of 35% strawberry extract, 7.5% cranberry extract, 2.5% raspberry seed extract, 2.5% elderberry extract and approximately 2.5% wild bilberry extract, which exhibited a significantly higher ORAC value than the other five combinations. The oxygen radical absorbance capacity of these various compositions of berry extracts are shown in FIG. 1.

Oxygen Radical Absorbing Capacity (ORAC)

The peroxyl-radical scavenging capacities of the berry compositions and of the GSPE composition were studied using the ORAC assay method describe above. Cranberry, elderberry, and raspberry seed compositions were observed to possess comparable ORAC values. The antioxidant capacities of these berry compositions were significantly lower than that of the other berry compositions studied. The ORAC values of the strawberry and GSPE compositions were higher than that of the cranberry, elderberry or raspberry seed, but significantly lower than that of the other compositions studied. Wild bilberry and wild blueberry compositions possessed the highest ORAC values. These values were comparable to the ORAC values of the two berry mixtures. The ORAC values of the various berry extract compositions of the present invention are graphically displayed in FIG. 1.

Anti-Angiogenic Properties

Tables 2 and 3 show tabular results of the effect of the berry compositions on inducible VEGF expression on the test cells. The data show that each of the berry compositions studied potently inhibited both H₂O₂-induced and TNFα-induced VEGF expression by the human keratinocytes. However, antioxidants such as GSPE, having a high ORAC value, or α-tocopherol did not influence inducible VEGF expression. This suggests that the observed effect of berry compositions was not dependent solely on their antioxidant properties. As illustrated in Table 5, pure flavonoids, such as ferrulic acid, catechin and rutin, shared the ability to suppress oxidant-inducible VEGF expression to some degree. Thus, it is evident that the flavonoid component of the berry compositions may have been responsible for at least part of the observed effect on inducible VEGF expression and release.

The present Example presents the first evidence showing that berry extracts potently inhibit inducible VEGF expression. Previously, certain antioxidants have been observed to have anti-angiogenic effects. However, our observation that GSPE possessing high antioxidant capacity failed to inhibit inducible VEGF expression suggests that the antioxidant property alone may not account for the observed anti-angiogenic effect. This is consistent with the findings that numerous plant-product constituents serve as potent regulator of several signal transduction pathways.

Our results with pure monomeric flavonoids present the first evidence that flavonoids may serve as potent inhibitors of inducible VEGF expression and that the flavonoid content of the berry compositions may have been responsible for the observed effect. Monomeric flavonoids account for less than 1% of GSPE, which may explain the observed inability of GSPE to inhibit inducible VEGF expression. In addition to their inhibitory effect on inducible VEGF expression, the berry compositions also impaired angiogenesis in vitro. This, suggests that other key events in angiogenesis, such as integrin function, may be sensitive to berry constituents. These observations provide a firm mechanism-based support to the contention that edible berries may provide a feasible diet-based approach to prevent the angiogenesis-related disorders such as cancer and inflammation.

Approximately 50% of the earth's population is infected with Helicobacter pylori, which has been implicated in the etiology of chronic gastritis and peptic ulcer, both in adults and children. Several oral antimicrobial agents have efficacy against H. pylori. Clarithromycin is a key component of many therapeutic regimens recommended for eradication of gastric H. pylori. Antibiotic treatment for H. pylori infection is often accompanied by side effects including development of resistance to antimicrobial agents, including clarithromycin.

Natural antioxidants might serve as novel therapeutic tools in alleviating H. pylori-induced oxidative damage. Several recent studies have demonstrated the inhibitory effect of cranberry juice and its constituents on H. pylori adhesion to human gastrointestinal cells. The exact mechanism of this inhibition is unclear at this time, but a plausible explanation may be the antioxidant property of cranberry juice, which is due to the presence of anthocyanins.

To discover the method and composition of the present invention, we evaluated the inhibitory effects of various berry extracts with and without clarithromycin, against a pathogenic strain of H. pylori (ATCC strain 49503), which is known to produce an 87 kDa cytotoxin responsible for gastric injury. This strain was chosen based on a previous study that demonstrated that in a tissue culture model, the greatest LDH (lactate dehydrogenase) leakage and superoxide anion production were caused by it compared to other strains of H. pylori.

In order to determine the efficacy of the method and composition of the present invention at preventing or inhibiting H. pylori, stock solutions of 5 mg/ml for clarithromycin were prepared in DMSO and stored at 40° C. Further dilutions of the drug were made in phosphate buffered saline for use in the bactericidal tests. Fresh solutions were prepared for each experiment.

Freeze dried cytotoxin-producing H. pylori strain ATCC 49503 was obtained from American Type Culture collection (Rockville, Md.). Freeze dried bacterial samples were re-dissolved in sterile Brucella broth and incubated at 370 C. for 30 minutes before being cultured on fresh blood agar plates.

Lennox broth (Fisher Chemicals) was used for growth of H. pylori. Trypticase soy agar (TSA) plates with 5% defibrinated sheep blood (BBL, Becton Dickinson, Md. 21152) were used for determining viable bacterial counts. Bacterial plates and culture tubes were incubated at 37° C., under microaerophilic conditions (oxygen 5%, carbon dioxide 10% and nitrogen 85%) in an incubator.

H. pylori was initially grown on 5% blood agar plates overnight. Cell suspensions were then prepared in 2 ml of PBS and diluted 10-fold. The various berry extracts mentioned above were incorporated into Lennox broth in concentrations of 0.25%, 0.5% and 1%, respectively, with control tubes having only the broth. 100 ul of the bacterial cell suspension was then added to each tube and incubated under microaerophilic conditions for 18 hours. Samples from each culture tube were then serially diluted, and 10 ul from the 10-7 dilution tube were plated on fresh 5% blood agar plates, which were then incubated under microaerophilic conditions for 18 hours and the number of colonies counted. Growth of H. pylori was confirmed by the CLO test. All experiments were conducted in triplicate.

The CLO test is a rapid urease test initially developed to detect the urease enzyme of H. pylori in gastric mucosal biopsies. It has also been used to detect urease production from H. pylori infection in tissue culture. Test slides were obtained from (Ballard Medical Products, Draper, Utah 84020). After 16 hours of incubation, bacterial cultures were tested for urease activity to confirm growth of H. pylori.

To determine the bactericidal effects of clarithromycin and berry extracts on H. pylori, each of the serially diluted experimental and control samples were incubated with 15 ug/ml of clarithromycin for 1 hour. Then, three replicates of 10 ul from each tube were plated on 5% blood agar plates and incubated under microaerophilic conditions for 18 hours and the number of colonies counted. Once again, growth of H. pylori was confirmed by the CLO test.

Statistical Analysis. Study results were entered into a database and analyzed using the ABSTAT software. Chi-square test was used to compare results at different concentrations. Level of significance was set at p<0.05.

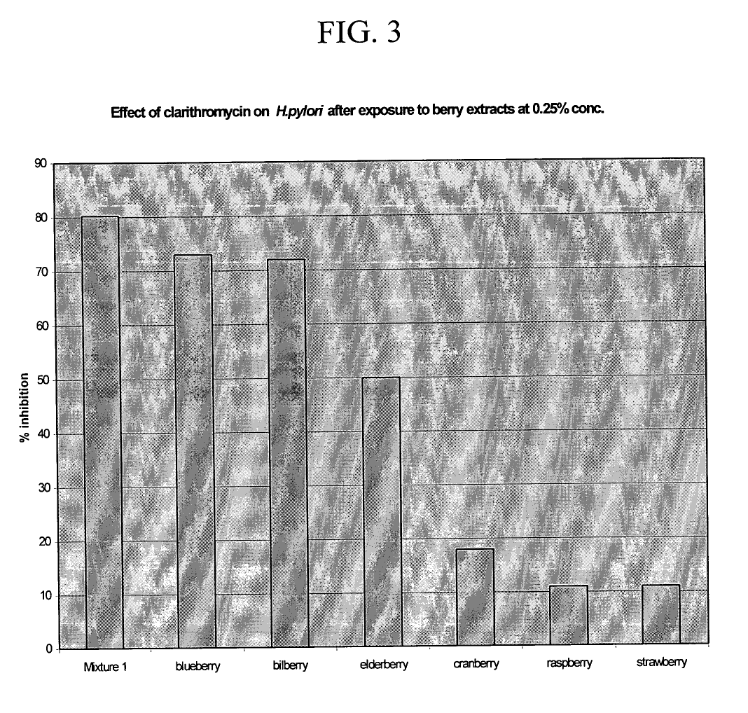

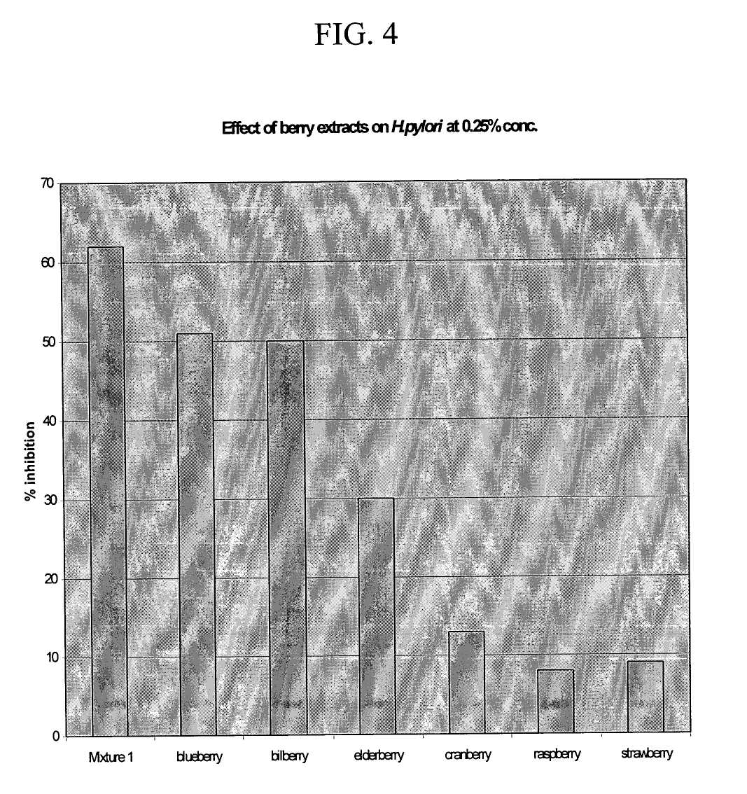

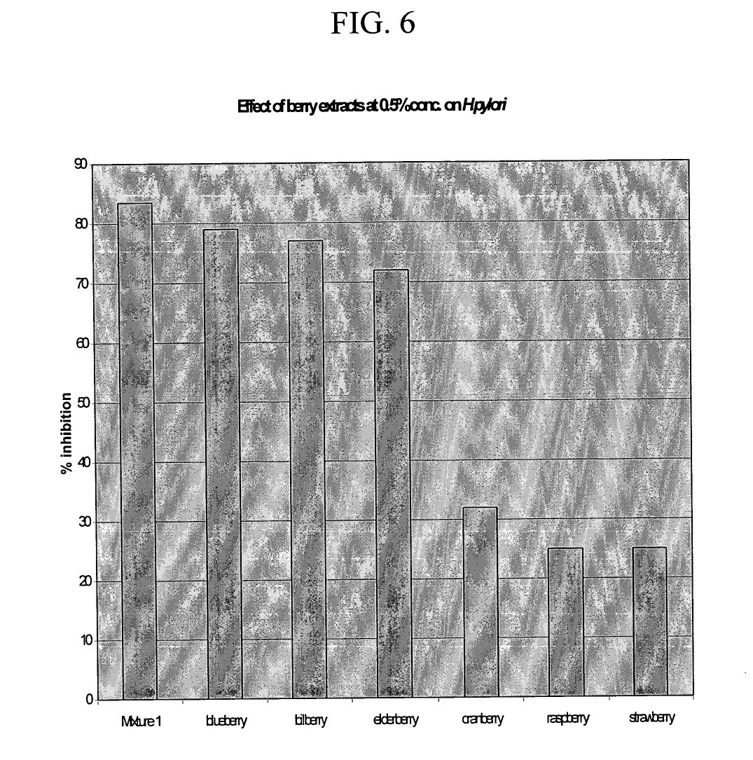

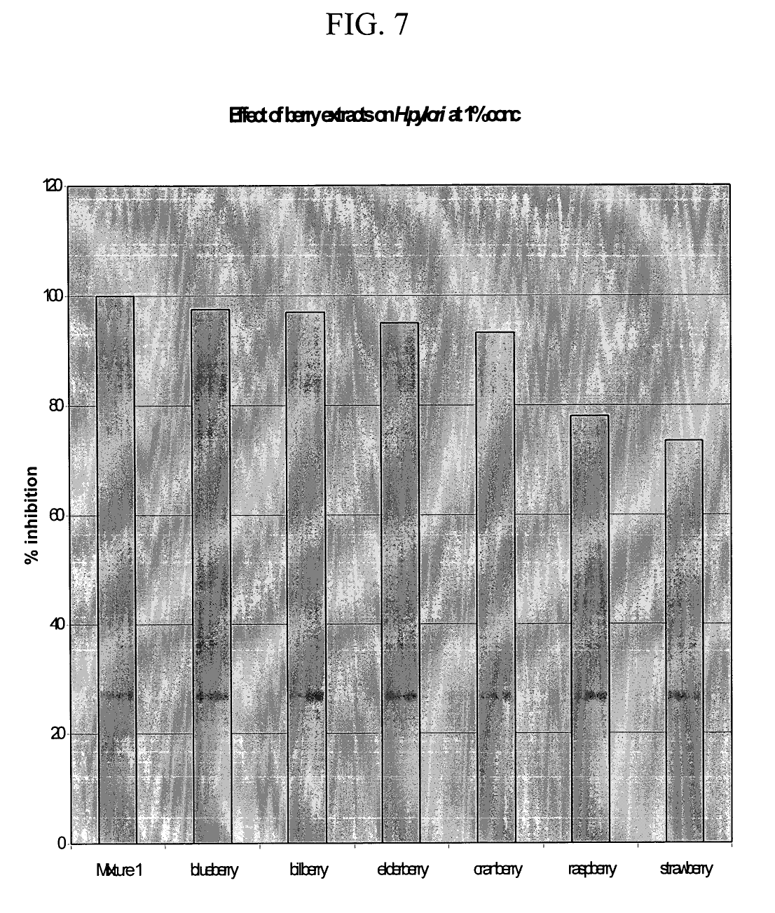

The in vitro bactericidal activities of the various berry extracts, with and without clarithromycin, against H. pylori are shown in FIGS. 3-8. All of the extracts at all concentrations tested inhibited the growth of H. pylori compared to controls, with maximal effects noted with Mixture 1. Even at the lowest concentration of 0.25%, significant inhibition of H. pylori was noted with elderberry (30%), wild bilberry (50%), wild blueberry (50.5%) and Mixture 1 (62%), as shown in FIG. 4. There was a concentration-dependent increase in inhibition with the higher concentrations of 0.5% and 1% of all the extracts, as shown in figures. 6 and 7. Modest increases in bactericidal effect were seen with the 0.5% concentration of strawberry, raspberry seed and cranberry extracts, compared to the increases noted for elderberry, wild bilberry, wild blueberry and Mixture 1, as shown in figure. 6. At the 1% concentration, all extracts showed>70% inhibition, with cranberry, elderberry, wild bilberry and wild blueberry extracts showing>90% inhibition, and Mixture 1 showing 100% inhibition, as shown in FIG. 7.

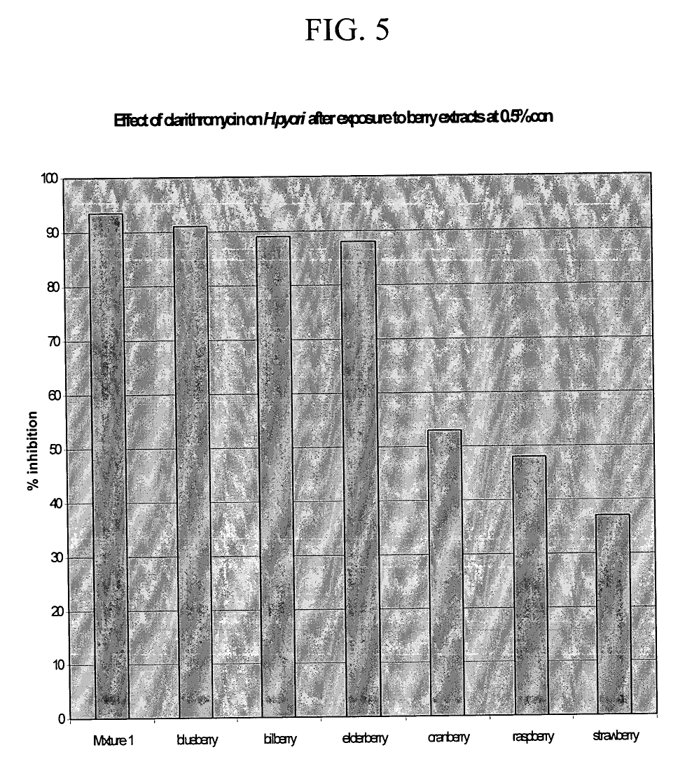

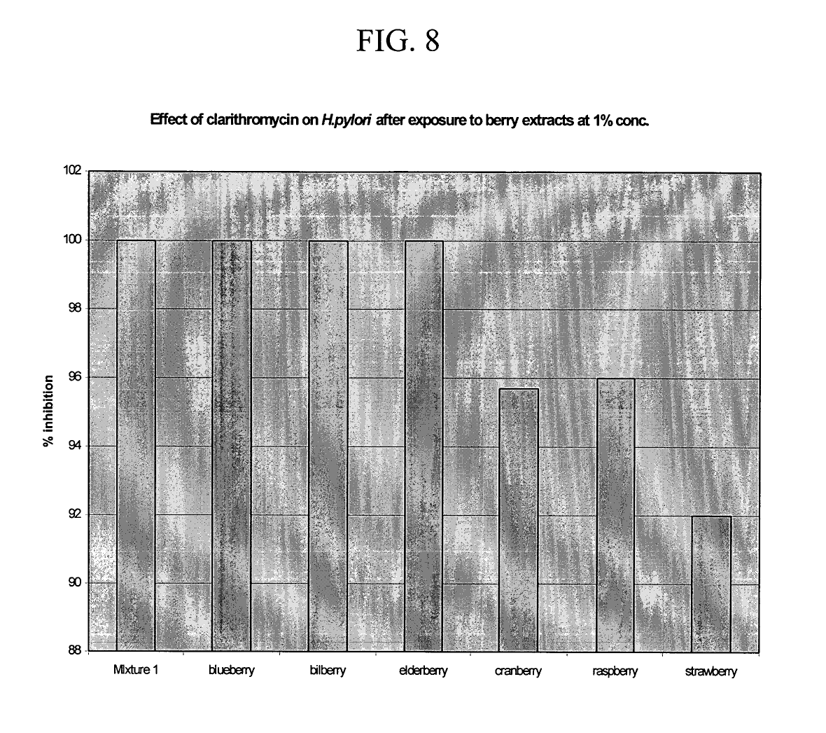

The addition of clarithromycin at the 0.25% concentration led to a significant increase in the bactericidal effects of the elderberry, wild bilberry, wild blueberry and Mixture 1 against H. pylori, as shown in FIG. 3. When clarithromycin was added to the 0.5% concentration, there was a significant increase in the inhibition of H. pylori by all the extracts tested, as shown in FIG. 5. Finally, when clarithromycin was added to the 1% concentration, >90% inhibition was noted for all extracts, with elderberry, wild bilberry, wild blueberry and OptiBerry showing 100% inhibition, as shown in FIG. 8.

As the results shown in FIGS. 3-8 indicate, there was a concentration-dependent inhibition of H. pylori noted, with the highest antibacterial activity noted at the 1% concentration of all extracts. It is also important to note that not all extracts had equivalent activity. Clearly, Mixture 1, as defined above, demonstrated maximal effects at all concentrations tested, but some of the others such as wild blueberry and wild bilberry had significantly better activity against H. pylori compared to raspberry seed, cranberry and strawberry, particularly at lower concentrations. Finally, an additive effect was noted with clarithromycin at all concentrations of the berry extracts tested, with maximal effects noted once again with Mixture 1. Therefore, the method and composition of the present invention use berry extracts to, among other things, effectively prevent or inhibit the growth of H. pylori, a known carcinogen and pathogen.

Although the invention has been disclosed in detail with reference only to the preferred embodiments, those skilled in the art will appreciate that additional methods and compositions can be made without departing from the scope of the invention.

I claim:

1. A method for preventing or inhibiting angiogenesis in a person, the method comprising:
identifying a person suffering from, or at risk of suffering from, angiogenesis; and
administering an effective amount of a composition comprising of more than one berry extract to the person, wherein the composition is sufficient to prevent or inhibit angiogenesis in the person.

2. A method of claim 1, wherein administering the composition to the person reduces the amount of VEGF expression in that person.

3. A method of claim 1, wherein the effective amount of the composition is from 18 mg/dose-270 mg/dose and the cytotoxicity of the composition is lower than 0.5 LDH units/liter.

4. A method of claim 1, wherein the composition contains more than one berry extract selected from the group consisting of blueberry extract, bilberry extract, cranberry extract, elderberry extract, raspberry extract and strawberry extract.

5. A composition of claim 4, wherein the blueberry extract is a wild blueberry extract, the bilberry extract is a wild bilberry extract and the raspberry extract is a raspberry seed extract.

6. A method of claim 1, wherein the composition by weight is approximately 50% blueberry extract, 35% strawberry extract, 7.5% cranberry extract, 2.5% raspberry extract, 2.5% elderberry extract and 2.5% bilberry extract.

7. A composition of claim 1, wherein the composition by weight is 50% wild blueberry extract, 35% strawberry extract, 7.5% cranberry extract, 2.5% raspberry seed extract, 2.5% elderberry extract and 2.5% wild bilberry extract.

8. A method of claim 1, wherein the composition by weight is approximately 50% blueberry extract, 25% strawberry extract, 12.5% bilberry extract, and 12.5% raspberry extract.

9. A composition of claim 1, wherein the composition by weight is 50% wild blueberry extract, 25% strawberry extract, 12.5% wild bilberry extract and 12.5% raspberry seed extract.

10. A method for providing a compound with a high oxygen radical absorbance capacity, the method comprising:
administering an effective amount of a composition comprised of more than one berry extract, wherein the composition has a higher oxygen radical absorbance capacity than the oxygen radical absorbance capacity of any one berry extract used in the composition.

11. A method of claim 10, wherein the composition has a higher oxygen radical absorbance capacity than an equal amount of GSPE.

12. A method of claim 10, wherein the composition has a lower cytotoxicity than an equal amount of GSPE.

13. A method of claim 10, wherein the effective amount of the composition is 18 mg/dose-270 mg/dose and the cytotoxicity of the composition is lower than 0.5 LDH units/liter.

14. A method of claim 10, wherein the composition has an oxygen radical absorbance capacity above 40 Troxol equivalents/gm fresh weight basis.

15. A method of claim 10, wherein the composition contains more than one berry extract selected from the group consisting of blueberry extract, bilberry extract, cranberry extract, elderberry extract, raspberry extract and strawberry extract.

16. A composition of claim 15, wherein the blueberry extract is a wild blueberry extract, the bilberry extract is a wild bilberry extract and the raspberry extract is a raspberry seed extract.

17. A method of claim 10, wherein the composition by weight is approximately 50% blueberry extract, 35% strawberry extract, 7.5% cranberry extract, 2.5% raspberry extract, 2.5% elderberry extract and 2.5% bilberry extract.

18. A composition of claim 10, wherein the composition by weight is 50% wild blueberry extract, 35% strawberry extract, 7.5% cranberry extract, 2.5% elderberry extract, 2.5% wild bilberry extract and 2.5% raspberry seed extract.

19. A method of claim 10, wherein the composition by weight is approximately 50% blueberry extract, 25% strawberry extract, 12.5% bilberry extract, and 12.5% is raspberry extract.

20. A composition of claim 10, wherein the composition by weight is 50% wild blueberry extract, 25% strawberry extract, 12.5% wild bilberry extract and 12.5% raspberry seed extract.

21. A method of claim 10, further comprising:
identifying a person who would benefit from using an antioxidant with a high oxygen radical absorbance capacity.

22. A method of claim 21, wherein the composition has a lower cytotoxicity than GSPE.

23. A method of claim 21, wherein the effective amount of the composition is 18 mg/dose-270 mg/dose and the cytotoxicity of the composition is lower than 0.5 LDH units/liter.

24. A method of claim 21, wherein the composition has a higher oxygen radical absorbance capacity than an equal amount of GSPE.

25. A method of claim 21, wherein the composition has a lower cytotoxicity than an equal amount of GSPE.

26. A method for preventing or inhibiting the growth of Helicobacter pylori in a person, the method comprising:
administering an effective amount of a composition comprised of more than one berry extract to a person, wherein the composition prevents or inhibits the growth of Helicobacter pylori more than any one berry extract used in the composition.

27. A method of claim 26, wherein the composition improves the ability of an antibiotic to prevent or inhibit the growth of Helicobacter pylori.

28. A method of claim 27, wherein the antibiotic is a range of 0.1%-5.0% concentration of clarithromycin.

29. A method of claim 27, wherein the antibiotic is preferably a range of 0.25%-1% concentration of clarithromycin.

30. A method of claim 26, wherein the effective amount of the composition is from 18 mg/dose-270 mg/dose and the cytotoxicity of the composition is lower than 0.5 LDH units/liter.

31. A method of claim 26, wherein the composition is prepared from more than one berry extract selected from the group consisting of blueberry extract, bilberry extract, cranberry extract, elderberry extract, raspberry extract and strawberry extract.

32. A composition of claim 31, wherein the blueberry extract is a wild blueberry extract, the bilberry extract is a wild bilberry extract and the raspberry extract is a raspberry seed extract.

33. A method of claim 26, wherein the composition by weight is approximately 50% blueberry extract, 35% strawberry extract, 7.5% cranberry extract, 2.5% raspberry extract, 2.5% elderberry extract and 2.5% bilberry extract.

34. A composition of claim 26, wherein the composition by weight is 50% wild blueberry extract, 35% strawberry extract, 7.5% cranberry extract, 2.5% elderberry extract, 2.5% wild bilberry extract and 2.5% raspberry seed extract.

35. A method of claim 26, wherein the composition by weight is approximately 50% blueberry extract, 25% strawberry extract, 12.5% bilberry extract, and 12.5% is raspberry extract.

36. A composition of claim 26, wherein the composition by weight is 50% wild blueberry extract, 25% strawberry extract, 12.5% wild bilberry extract and 12.5% raspberry seed extract.

37. A method of claim 26, further comprising:
identifying a person who would benefit from using a composition that prevents or inhibits the growth of Helicobacter pylori.

38. A method of claim 37, wherein the effective amount of the composition is from 18 mg/dose-270 mg/dose and the cytotoxicity of the composition is lower than 0.5 LDH units/liter.

39. A composition that prevents or inhibits angiogenesis or Helicobacter pylori or acts as a powerful antioxidant in the human body, the composition comprising:
selecting more than one berry extract selected from the group consisting of blueberry extract, bilberry extract, cranberry extract, elderberry extract, raspberry extract, and strawberry extract, wherein the berry extracts are selected and proportioned relative to all the selected berry extracts to provide a composition that effectively prevents or inhibits angiogenesis or Helicobacter pylori or has a high oxygen radical absorbance capacity.

40. A composition of claim 39, wherein the blueberry extract is wild blueberry extract, the bilberry extract is a wild bilberry extract and the raspberry extract is a raspberry seed extract.

41. A composition as in claim 39, wherein the group of berry extracts further consists of blackberries, dewberries, boysenberries, loganberries, youngberries, currants, gooseberries, juniper berries, huckleberry, elderberry, thimbleberry, blackcap berries, mountain ash berries, salmonberry and other similar berries.

42. A composition of claim 39, wherein the composition results in less VEGF being expressed in a person who absorbs the composition.

43. A composition of claim 39, wherein the composition has a lower cytotoxicity than an equal amount of GSPE.

44. A composition of claim 39, wherein the effective amount of the composition is from 18 mg/dose-270 mg/dose

44. A composition of claim 39, wherein the composition has a higher oxygen radical absorbance capacity than both an equal amount of GSPE and an equal amount of any one berry extract.

45. A composition of claim 39, wherein the composition has an oxygen radical absorbance capacity above 40 Troxol equivalents/gm fresh weight basis.

46. A composition of claim 39, wherein approximately 50% of the weight of the composition is blueberry extract, approximately 35% of the weight is strawberry extract, approximately 7.5% of the weight is cranberry extract, approximately 2.5% of the weight is raspberry extract, approximately 2.5% of the weight is elderberry extract and approximately 2.5% of the weight is bilberry extract.

47. A composition of claim 39, wherein the composition comprises by weight 50% wild blueberry extract, 35% strawberry extract, 7.5% cranberry extract, 2.5% elderberry extract, 2.5% wild bilberry extract and 2.5% raspberry seed extract.

48. A composition of claim 39, wherein approximately 50% of the weight of the composition is blueberry extract, approximately 25% of the weight is strawberry extract, approximately 12.5% of the weight is bilberry extract, and approximately 12.5% of the weight is raspberry extract.

49. A composition of claim 39, wherein approximately 50% of the weight of the composition is wild blueberry extract, approximately 25% of the weight is strawberry extract, approximately 12.5% of the weight is wild bilberry extract, and approximately 12.5% of the weight is raspberry seed extract.
